(19) United States
(12) Patent Application Publication
(10) Pub. No.: US 2016/0015560 A1
(43) Pub. Date: Jan. 21, 2016

(54) METHODS TO IMPROVE THE PERFORMANCE OF THERMOELECTRIC HEATING/COOLING DEVICES

(71) Applicant: Innovation Alley, LLC, Lyndhurst, OH (US)

(72) Inventors: Farhad Bahremand, Seven Hills, OH (US); Brad A. Pulver, Shaker Heights, OH (US); Bahman Guyuron, Lyndhurst, OH (US); Jamie Horvath, Twinsburg, OH (US)

(21) Appl. No.: 14/802,506

(22) Filed: Jul. 17, 2015

Related U.S. Application Data
(60) Provisional application No. 62/025,653, filed on Jul. 17, 2014.

(51) Int. Cl. A61F 7/00; H01L 35/30

(52) U.S. Cl. CPC A61F 7/007; H01L 35/30; A61F 2007/0054

(57) ABSTRACT

The present disclosure provides methods to improve the performance of heat exchangers used in thermoelectric cooling/heating devices, wherein improved heat conduction between heat exchanger and thermal exchange fluid is accomplished. Additionally, a method is disclosed to minimize the necessary delay used to protect the thermoelectric modules against thermal shock when switching from heat to cold, or vice versa. Thermal shock can damage thermoelectric modules when the direction of current passing through the modules is instantly switched.

### RELATED APPLICATIONS

This application claims the benefit of U.S. Provisional Application No. 62/025,653 filed Jul. 17, 2014, which is hereby incorporated herein by reference.

### FIELD OF INVENTION

The present invention relates generally to thermoelectric heaters and coolers, and more particularly to improvements in the performance of heat exchangers used in thermoelectric cooling/heating devices.

### BACKGROUND

Thermal therapy is the practice of applying heat and/or cold to tissue to reduce swelling/inflammation, to decrease pain, promote healing, increase blood flow, alleviate aches, calm stress points, and/or just for general relaxation. The thermal effect (i.e., heat or cold) can be accomplished by the heating or cooling effect of a therapy-providing fluid (e.g., water, oil) in thermal contact with the relevant tissue. To this end, a tissue-interacting device (containing the therapy providing fluid) can be strapped or otherwise held in contact with the relevant areas of the therapy-receiving person's body.

### SUMMARY OF INVENTION

The present disclosure provides methods to improve the performance of heat exchangers used in thermoelectric cooling/heating devices, wherein improved heat conduction between heat exchanger and thermal exchange fluid is accomplished. Additionally, a method is disclosed to minimize the necessary delay used to protect the thermoelectric modules against thermal shock when switching from heat to cold, or vice versa. Thermal shock can damage thermoelectric modules when the direction of current passing through the modules is instantly switched.

To address the rapid switching issue, a delay may be generated by a control system, which automatically engages when the polarity of the applied DC voltage is switched. During the delay period, no power may be applied to the thermoelectric modules. The duration of the delay may be programmed to be sufficiently high to allow the thermoelectric modules return to about ambient temperature. The problem with this approach is that the fixed duration delay will still engage when switching the polarity of the applied DC voltage even when the differential temperature between the two plates is quite low and close to ambient temperature.

The present disclosure addresses this problem by using the same thermoelectric module as an electrical generator. Thermoelectric modules can convert thermal energy to electricity. When the two plates of a thermoelectric module are held at two different temperatures, a voltage is generated at the terminals of the thermoelectric module. The magnitude of the voltage depends on the differential voltage. Therefore, a higher differential temperature results in a higher generated voltage.

Therefore, it is possible to take advantage of the generated voltage to detect the actual differential temperature present at the plates of the thermoelectric module before switching the polarity. For this purpose, the temperature controller may be programmed to turn off the thermoelectric module at the polarity switching time. The temperature controller may then sample the voltage generated by the thermoelectric module in a loop until the voltage drops below a threshold indicating a safe differential temperature at which point polarity switching can take safely take place.

Another method of improving the efficiency of thermoelectric devices is by improving thermal conduction between the thermoelectric device(s) and the medium being heated or cooled. Two methods are described herein to improve the thermal conduction in a heat exchanger, where the thermal energy to the heat exchanger is applied by one or more TEC modules.

Controlling temperature of a fluid is one useful application for thermoelectric modules. In such an application, the fluid to be temperature-controlled passes through fluid conduits of a conductive solid body mounted on one plate of the thermoelectric module. The present disclosure addresses two methods to improve the thermal conductance between the thermoelectric modules and the liquid flowing in the fluid conduit.

According to one aspect of the invention, a thermal therapy system includes a tissue-interacting device to provide thermal therapy to a structure of a therapy-receiving person's body; a fluid-manipulating device which heats/cools the therapy-providing fluid including a pump for motivating the circulation of the therapy-providing fluid through the system and a heat exchanger for heating/cooling the therapy-providing fluid; tubing, and associated fittings, between the tissue-interacting device and the fluid-manipulating device; and an electrical control for controlling the temperature of the therapy-providing fluid, wherein the heat exchanger comprises a heat sink, a block through which the therapy-providing fluid flows, and one or more thermoelectric devices placed in thermal contact with the sink and the block, wherein the block includes a monolithic plate having a fluid passage defined therein by machined surfaces therein and by one or more plugs.

Optionally, the one or more plugs are fiberglass.

Optionally, the one or more plugs are epoxied to the plate.

Optionally, the fluid passage is serpentine and the one or more plugs fit into respective lands machined into side surfaces of the plate and define outer bends of the serpentine passage.

Optionally, the monolithic plate is copper.

Optionally, the serpentine fluid passage has a circular cross-section.

According to another aspect, a block for a heat exchanger includes a monolithic plate having a serpentine fluid passage contained therein, the fluid passage having a straight portion with sidewalls defined by machined inner surfaces of the plate and a bend portion with sidewalls defined by machined inner surfaces of the plate and a sidewall defined by a plug.

Optionally, the plug is fiberglass.

Optionally, the plug is epoxied to the plate in a machined recess of a long edge of the plate.

Optionally, the monolithic plate is copper.

Optionally, the straight portion of the serpentine fluid passage has a circular cross-section.

Optionally, the sidewall defined by the plug is on an exterior bend of the bend portion.

According to another aspect, a method of making a block for a heat exchanger includes drilling a series of through-holes into monolithic plate, the through-holes extending from a first edge of the plate to an opposite second edge of the plate; side milling a first recess in one of the edges between two adjacent through-holes, the recess having a first depth; side milling a second recess in the same one of the edges between and around the two adjacent through-holes, the recess having a second depth and the second depth being shallower than the first depth; and fitting a plug into the second recess and adhering the plug to the plate.

Optionally, the method includes side milling a third recess in the same one of the edges around the second recess, the third recess having a third depth, the third depth being shallower than the second depth.

Optionally, the edges are long edges of the plate, and wherein the through-holes extend parallel to short edges of the plate.

Optionally, the method includes fitting rigid tubing nubs to inlet and outlet openings of the plate.

Optionally, the plug is fiberglass.

Optionally, the monolithic plate is copper.

According to another aspect a method of controlling a thermoelectric module includes sampling generated voltage of the thermoelectric module; comparing the generated voltage to a predetermined safe threshold value; and determining if the differential temperature between the plates has fallen below a safe temperature threshold based on the comparing.

Optionally, the method includes switching a polarity of an applied DC voltage when the determining step determines that the differential temperature between the plates has fallen below a safe temperature threshold, and not switching the polarity of the applied DC voltage when the determining step determines that the differential temperature between the plates has not fallen below a safe temperature threshold; and applying a DC voltage with switched polarity.

Optionally, the method includes receiving a signal to switch a polarity of an applied DC voltage being applied to the thermoelectric module.

Optionally, the method includes stopping an applied DC voltage being applied to the thermoelectric module.

According to another aspect, a method of controlling a thermoelectric module includes receiving a signal to switch a polarity of an applied DC voltage being applied to the thermoelectric module; stopping the applied DC voltage being applied to the thermoelectric module; sampling generated voltage of the thermoelectric module; comparing the generated voltage to a predetermined safe threshold value; determining if the differential temperature between the plates has fallen below a safe temperature threshold based on the comparing; switching a polarity of an applied DC voltage when the determining step determines that the differential temperature between the plates has fallen below a safe temperature threshold, and not switching the polarity of the applied DC voltage when the determining step determines that the differential temperature between the plates has not fallen below a safe temperature threshold; and applying a DC voltage with switched polarity.

According to another aspect, a thermoelectric heat exchanger includes one or more thermoelectric modules; a heat exchanging block thermally coupled to the one or more thermoelectric modules; tubing defining a fluid passageway arranged in a spiral within the block, wherein an innermost loop of the tubing has a radius corresponding to a minimum bending radius of the tubing.

According to another aspect, a thermoelectric heat exchanger includes one or more thermoelectric modules; a heat exchanging block thermally coupled to the one or more thermoelectric modules; a first layer of tubing defining a fluid passageway arranged in within the block; and a second layer of tubing defining a fluid passageway arranged in within the block.

Optionally, the first and second layers of tubing are in fluid communication with one another.

The foregoing and other features of the invention are hereinafter described in greater detail with reference to the accompanying drawings.

### BRIEF DESCRIPTION OF THE DRAWINGS

FIG. 1 is a schematic view of a thermal therapy system, the system including a fluid-manipulating device and a tissue-interacting device;

FIG. 1B is a top perspective view of the fluid-manipulating device;

FIG. 1C is a bottom perspective view of the fluid-manipulating device;

FIG. 2 shows an exemplary tissue-interacting device;

FIG. 3 is a full cross sectional view of the fluid-manipulating device;

FIG. 4A shows an exploded view of a heat exchanger;

FIG. 4B shows an exploded view of a heat exchanger;

FIG. 4C shows an exploded view of a heat exchanger;

FIG. 5 shows electrical schematic for use in a thermoelectric heat exchanger;

FIG. 6 shows a method of controlling a thermoelectric heat exchanger;

FIG. 7 shows an arrangement of tubing for use in a heat exchanger;

FIG. 8 shows an arrangement of tubing for use in a heat exchanger;

FIG. 9 shows an arrangement of tubing for use in a heat exchanger;

FIG. 10 shows an arrangement of tubing for use in a heat exchanger;

FIG. 11 shows a solid model of the aluminum heat exchanger with scroll copper tubing embedded;

FIG. 12 shows a solid model of a two layer tubing pattern;

FIG. 13 shows a copper plate configured for two thermoelectric modules;

FIG. 14 shows a monolithic copper plate to be used in a block after some machining operations;

FIG. 15 shows a monolithic copper plate to be used in a block after some machining operations;

FIG. 16 shows a monolithic copper plate to be used in a block after some machining operations;

FIG. 17 shows an end view of the machined copper plate for use in a block;

FIG. 18 shows an end view of the machined copper plate for use in a block;

FIG. 19 shows a detailed view of the machined copper plate for use in a block;

FIG. 20 shows a top view of the machined copper plate for use in a block;

FIG. 21 shows a top-view cross-section of the machined copper plate for use in a block;

FIG. 22 shows a side view of the machined copper plate for use in a block;

FIG. 23 shows a transverse cross section of the machined copper plate for use in a block;

FIG. 24 shows another exemplary machined copper plate for use in a block;

FIG. 25 shows the machined copper plate for use in a block with installed plugs;

FIG. 26 shows the machined copper plate for use in a block with installed plugs and drilled inlet and outlet;

FIG. 27 shows the machined copper plate for use in a block with installed plugs and tubing;

FIG. 28 shows a front view of an exemplary plug;

FIG. 29 shows a side view of an exemplary plug;

FIG. 30 shows a side view of an exemplary tubing connection;

FIG. 31 shows a top view of an exemplary tubing connection;

FIG. 32 shows another exemplary plate for use in a block;

FIG. 33 shows an exemplary plug for use with the plate of FIG. 32; and

FIG. 34 shows an exemplary plate with exemplary plug installed.

### DETAILED DESCRIPTION

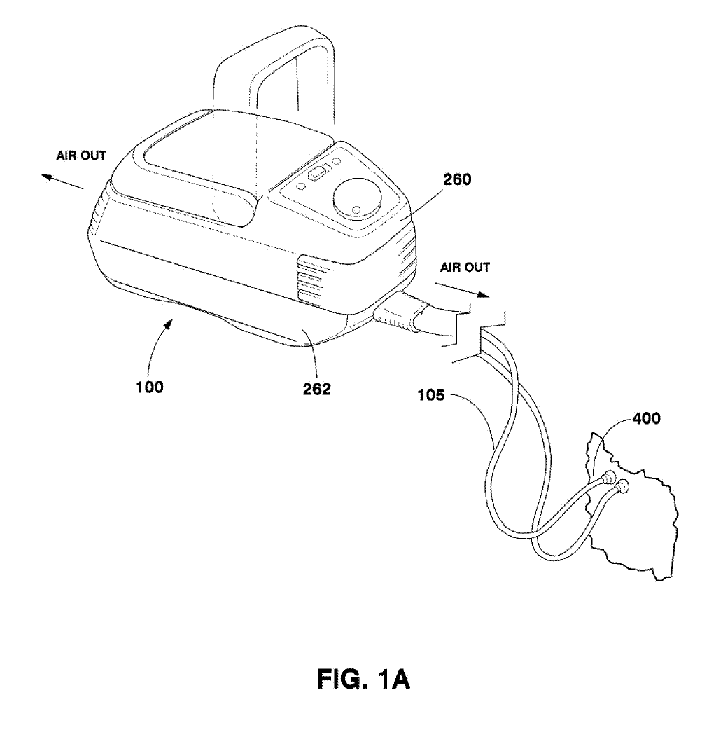

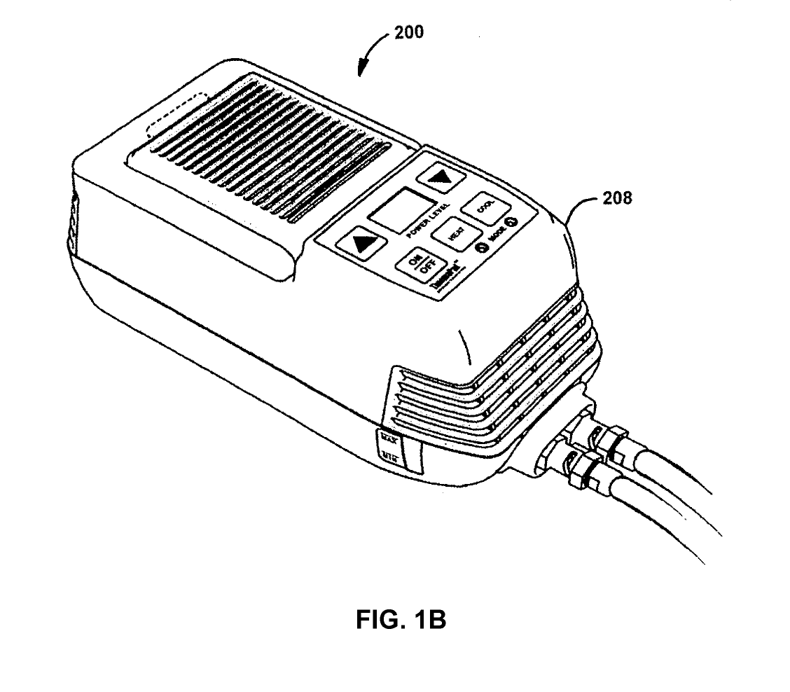

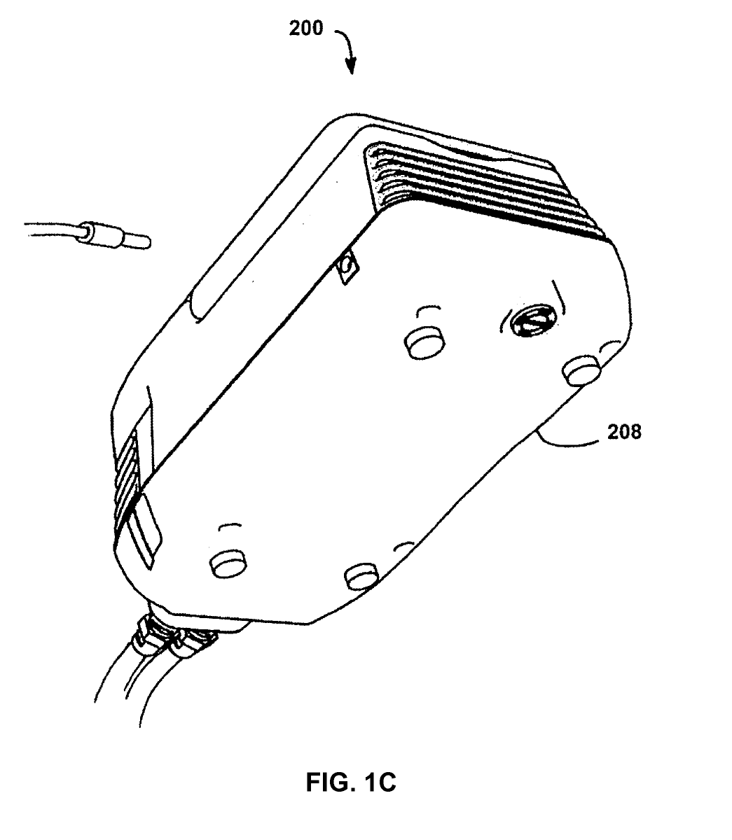

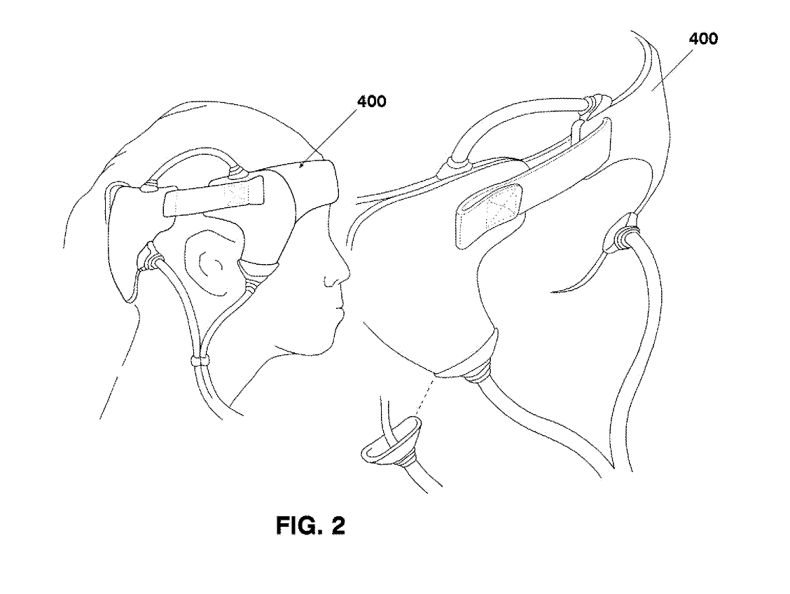

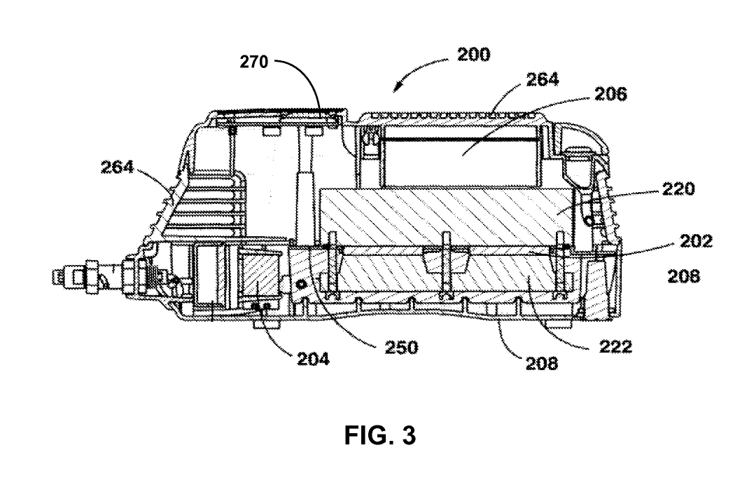

Referring now to the drawings, and initially to FIGS. 1A-C and FIGS. 2-3, a thermal therapy device 100 is schematically shown. The thermal therapy device 100 comprises a fluid-manipulating device 200, a tissue-interacting device 400, and plumbing 105 establishing fluid circulation paths therebetween. The fluid-manipulating device 200 heats/cools the therapy-providing fluid (e.g., water, oil) and pumps it through fluid channels in the tissue-interacting device 400. The tissue-interacting device 400 is placed in contact with the appropriate areas of the therapy-receiving person's body so that fluid passing through the channels can thermally interact therewith.

The fluid manipulating device 200 can comprise a heat exchanger 202, a pump 204, a fan 206, and a housing 208 enclosing these components. The heat exchanger 202 heats/cools the therapy-providing fluid, and the pump 204 circulates the therapy-providing fluid through the system 100. The fan 206 interacts with the heat exchanger's heat sink (sink 220, introduced below). The fluid-manipulating device 200 can be powered by direct 12 v (e.g. car power adaptor) or via an AC/DC converter. Alternatively, the device could be powered by a battery pack (either single use or rechargeable).

The housing 208 can include a top portion 260 and a bottom portion 262. Vents 264 (e.g., slats, screens, etc.) can be provided to permit air to be pulled into the fan 206 and then expelled as it blows across the heat sink 220. For example, as illustrated, air is pulled into the housing via top vents and expelled via side vents, but any compatible air path may be used.

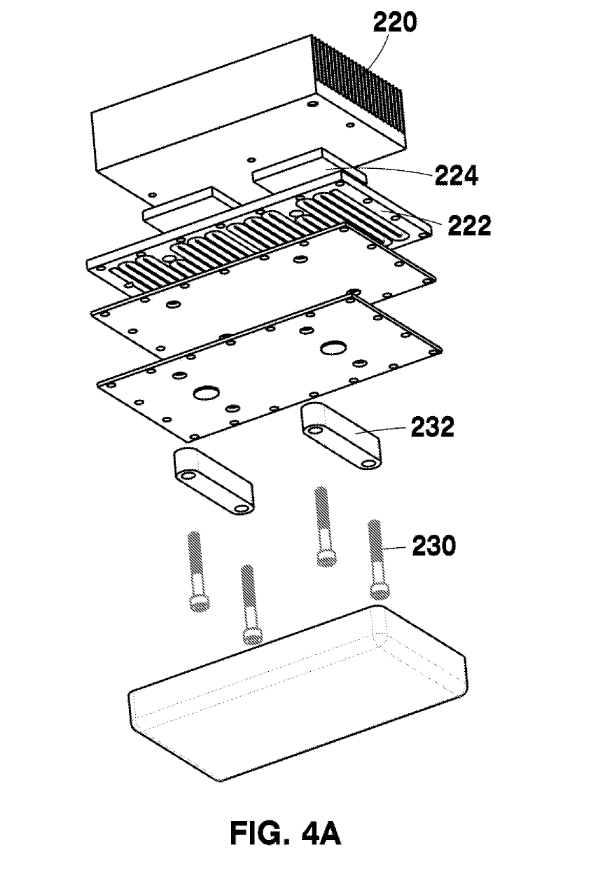

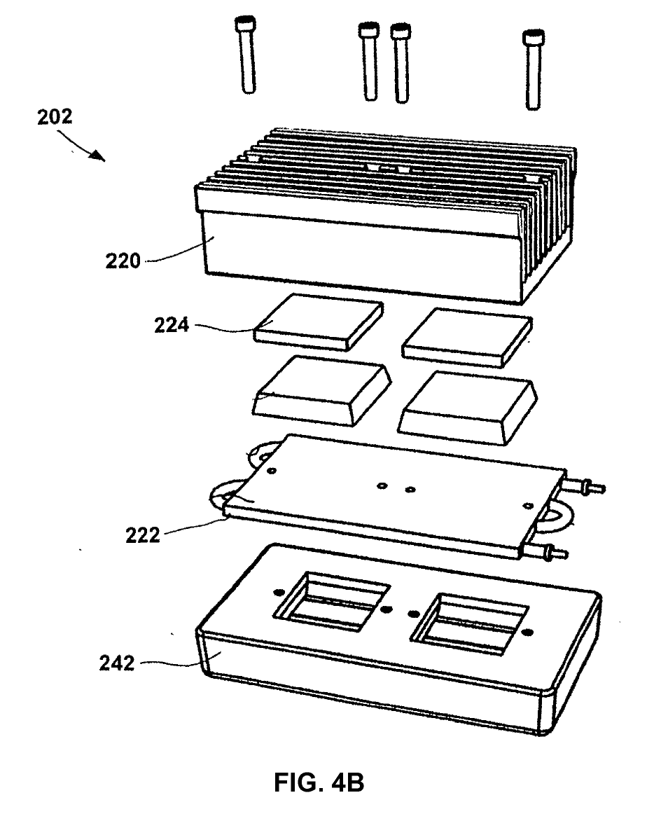

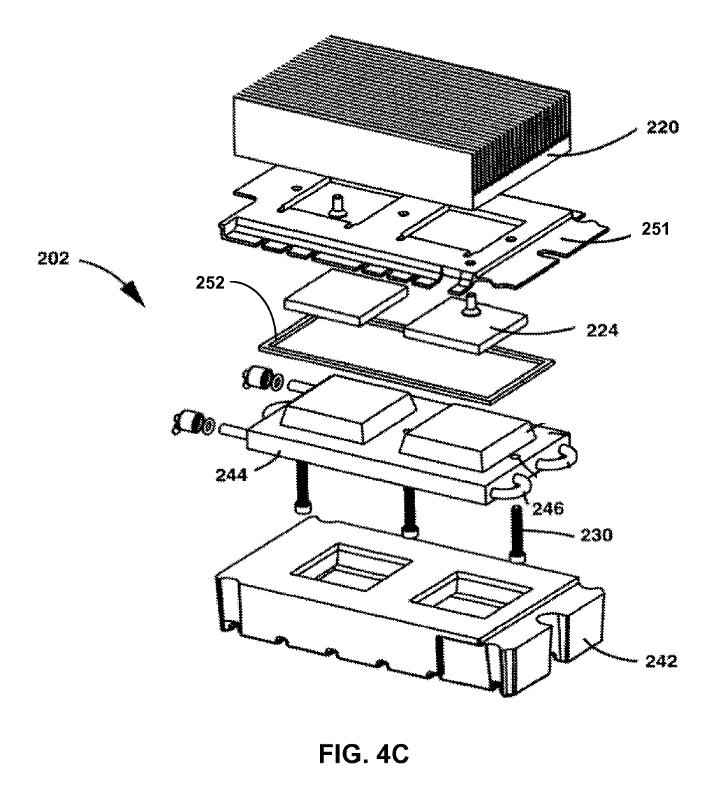

The heat exchanger 202 (shown in detail in FIGS. 4A, 4B and 4C) can comprise a heat sink 220, a fluid-passthrough portion 222 (e.g., a block or a cold plate), and thermoelectric module(s) 224 (e.g., Peltier devices).

The fluid-passthrough portion 222 may be a block secured to the heat sink 220 (e.g., with screws 230) with the thermoelectric modules 224 situated therebetween. Clamp bars 232 can be used. The block 222 comprises flow passages therethrough which form part of the fluid circulation path.

As an example, the fluid-passthrough portion 222 may include a cold plate 244 die cast around copper tubing 246 (e.g. in a serpentine configuration) that creates the channels 240. Alternative passthrough portions are described in more detail below. Spacers 248 can be situated between the thermoelectric modules 224 to act as insulation pads when fluid-manipulating device 200 is operating in the cooling mode. The spacers 248 can be separate elements (FIG. 4B) or they can be integrally formed with the passthrough portion 222 (FIG. 4C). In either or any event, the thermoelectric modules 224 interface with the heat sink 220.

The heat exchanger 202 can further comprise a mounting plate 251 and a gasket 252 forming a seal around the thermoelectric modules 224. Machine screws 230, passing through clearance holes in the cold plate 240 can fasten these components. Components are clamped together under pressure, and the machine screws fastened into aligning tapped holes in the heat sink 220. The mounting plate is fastened to the outer periphery of the heat sink 220 and screws the heat exchanger 202 to the housing 208.

The passthrough portion 222 and thermoelectric modules 224 may be insulated using polystyrene, polyurethane, or similar insulating material. These materials may be pre-cut to shape, formed to shape, or poured/molded directly in place. Thermal grease may be used on the tops/bottoms of the thermoelectric modules 224 to ensure good contact with other heat-exchanger components and thereby insure efficient temperature conductivity. Insulation 242 may be provided.

The flow rate produced by the pump 204 may be pre-set or user controlled to achieve varying temperature ranges. The type of pump used may be a diaphragm pump, peristaltic pump, etc. In order to control the flow rate, a regulating valve connected to the pump may be used.

The fan 206 can be placed in direct contact with the heat sink and configured to direct airflow into the heat sink or to pull air away from heat sink, depending upon the desired thermal conditions.

Optionally, a reservoir 250 could be incorporated into the system 100. (See FIG. 3.) This reservoir 250 could be cooled and/or heated using additional thermoelectric devices 224 or could be used to simply hold fluid before or after it is passed through the block/plate 222. The reservoir 250 can be comprised of a body and lid portion, joined together by ultrasonic weld or solvent to form a leak-proof vessel. The sides of the reservoir body fit into receiving slots in the housing 208, positioning the reservoir within. Two lengths of tubing to fill the reservoir fit over protrusions in the reservoir lid portion and extend rearward to connect to the fill port detail integral to the top housing. One of these lengths provides a conduit for fluid to enter into the reservoir, while the other provides a conduit for air to exit out from the reservoir. A urethane rubber-fill cap creates a watertight seal over the fill port when not in use. A length of tubing connects to a further protrusion in the reservoir lid and serves as the fluid supply conduit to the diaphragm pump.

As shown in FIG. 3, side vents can be trapped between the housing portions 260/262. A PCB 270 is fastened to the underside of the top housing and includes a LCD used to operate the device. The window of the LCD aligns with an opening in the top housing and a read through window in the affixed membrane switch. Headers located on the bottom face of the PCB receive plugs with leads to all electrical components within the device providing easy assembly. A temperature sensor is also incorporated into the PCB, and provides a means by which to regulate the system performance in both heating and cooling modes to keep tissue-interacting devices from becoming too hot or too cold. An additional electronic function designed into the PCB is an automatic power delay when switching from one mode to the other. This protects the thermoelectric devices from degradation that otherwise may occur when switching polarities too quickly, the means by which one mode changes to the other. A plastic handle features protruding side clips that snap assemble to the outside of the top housing and provide a fulcrum point at which the handle can swing up vertically for carrying the device.

The advent of thermoelectric modules, also called a Peltier module, Peltier device, Peltier heat pump, solid state refrigerator, or thermoelectric cooler (TEC), offers a viable alternative cooling method to compressors used in cooling applications, and also a viable alternative to heating elements used in heating applications. The thermoelectric modules create new ability for both cooling and heating in the same application with no need for either compressors or heating elements. The primary advantages of a Peltier device compared to a vapor-compression refrigerator are its lack of moving parts or circulating liquid, very long life, invulnerability to leaks, small size and flexible shape. Its main disadvantage is high cost and poor power efficiency.

By applying a DC voltage to a thermoelectric module, heat will be moved in the module from one plate to the other. Moving heat from one plate to the other implies that heat is removed from one plate to the other plate, and in effect, one plate gets cold while the other plate gets hot. The effect is reversed if the polarity of the applied DC voltage is reversed, and this is how a thermoelectric module can be used for both heating and cooling. Thermoelectric modules are available in various physical shapes and sizes, and are offered in broad power ratings. The thermal capacity of a thermoelectric module depends on the magnitude of the current passing through the module as well as the power rating of the thermoelectric module itself. Since the thermal energy generated or removed from the plates depends on the magnitude of current applied to the module, it is possible to design temperature controller devises by regulating the current applied to the thermoelectric module. In a typical application, the object that needs to be temperature controlled is connected to one plate, while a heat sink is connected to the other plate to remove the “unwanted” thermal energy. To increase the total thermal capacity, it is possible to connect two or more modules in series or parallel.

The heat exchanger, via user controlled switches and/or dials, will be able to operate in either cold or hot mode. When in cold mode, the surface of the thermoelectric device in contact with the block will be cold. By changing polarity, the device can switch to hot mode which results in the surface of the thermoelectric device in contact with the block to become hot.

In a temperature-controlled system where the same thermoelectric module is used for both cooling and heating, rapid reversing of the applied DC voltage will permanently damage the thermoelectric module, reduce its performance, or shorten its effective life. When a thermoelectric module is used to generate heat on one of its plates, the semiconductor PN junctions connecting the two plates are hot one end and cold on the other. Rapid reversing of the applied DC voltage will create an instant rate of expansion-contraction on the PN junctions as well as on the plates. This can permanently break the PN junction or crack the plates.

Optionally, an electrical control could be introduced that prevents the user from instantly switching polarity, e.g., if the user activates the polarity switch, power to the devices will be turned off for five minutes to allow the system temperature to acclimate more closely to ambient before turning the power back on. This type of control will prevent the devices from being shocked by rapid and dramatic swings in temperature. Optionally, electrical controls 270 could be introduced that limit the temperature that the liquid can achieve. This could include a high temperature and/or low temperature control. Moreover, regardless of the mode of operation, the user may be able to control the intensity of the temperature via a control on the housing. The device may optionally include controls to maintain specific temperature ranges. Additionally, an LCD readout could be incorporated to display data such as actual temperature, desired temperature, etc.

To address the rapid switching issue, a delay may be generated by the control system, which automatically engages when the polarity of the applied DC voltage is switched. During the delay period, no power is applied to the thermoelectric modules. The duration of the delay is programmed to be sufficiently high to allow the thermoelectric modules return to about ambient temperature. A problem with this approach is that the fixed duration delay will still engage when switching the polarity of the applied DC voltage even when the differential temperature between the two plates is quite low and close to ambient temperature.

Exemplary methods address this problem by using the same thermoelectric module as an electrical generator. Thermoelectric modules can convert thermal energy to electricity. When the two plates of a thermoelectric module are held at two different temperatures, a voltage is generated at the terminals of the thermoelectric module. The magnitude of the voltage depends on the differential voltage, therefore, the higher the differential temperature, the higher the generated voltage.

Exemplary methods take advantage of the generated voltage to detect the actual differential temperature present at the plates of the thermoelectric module before switching the polarity. For this purpose, the temperature controller is programmed to turn off the thermoelectric module at the polarity switching time, and to sample the generated voltage in a loop until the generated voltage drops below a threshold indicating a safe differential temperature at the plates, safe to make polarity switching.

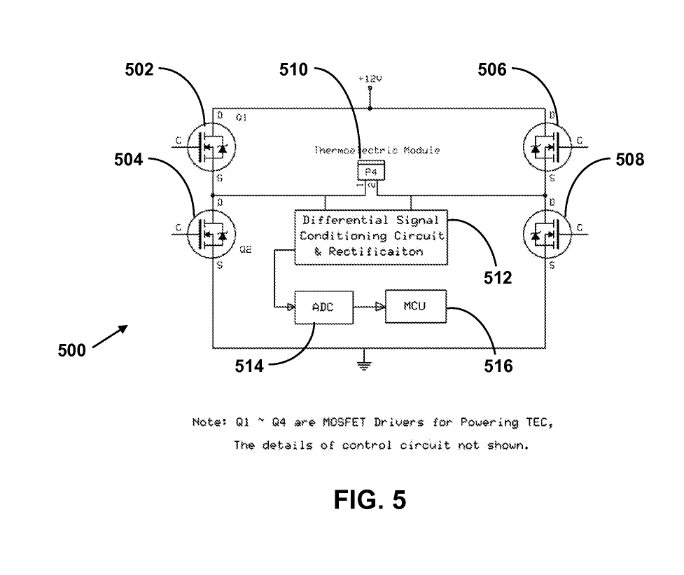

Referring initially to FIG. 5, the schematic of the electrical system 500 shows the MOSFET drivers 502, 504, 506, 508 used to power and switch the thermoelectric module(s) 510. Further, a signal conditioning module 512 may be included to scale and or otherwise condition the generated voltage and may feed the conditioned voltage to an analog to digital converter (ADC) 514, so that it can be processed by a digital control unit 516, for example, a microcontroller unit (MCU). It should be evident to those skilled in the art after understanding this disclosure, however, that aspects of the invention may be implemented in other ways, for example, through an analog control circuit.

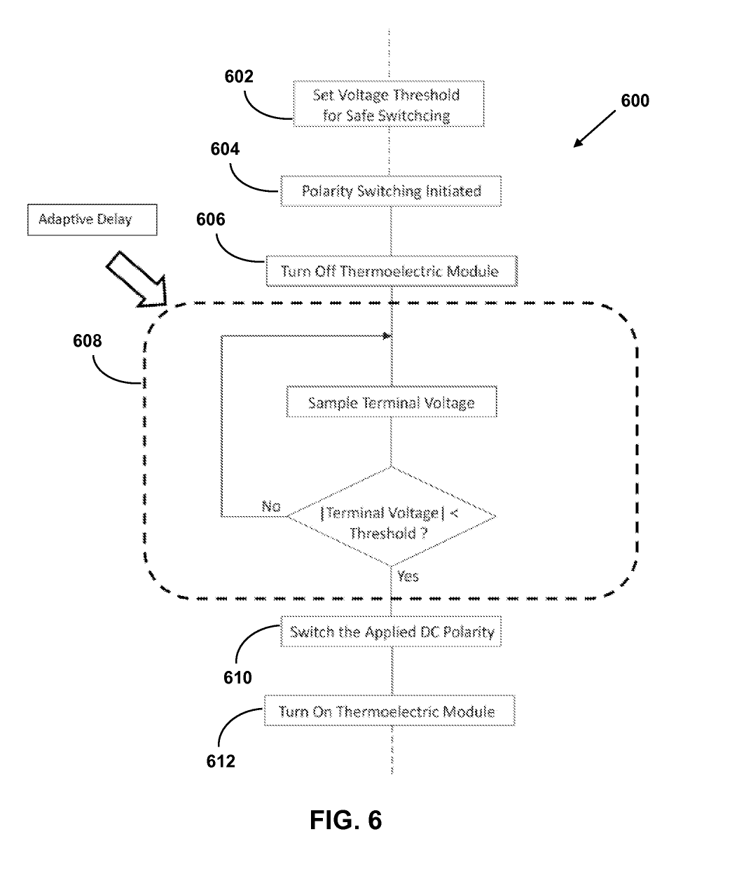

FIG. 6 depicts a flowchart that shows the high level routine 600 for sampling the thermoelectric module's generated voltage to determine if the differential temperature between the plates has fallen below a safe threshold so that the polarity of the applied DC voltage can be switched.

At block 602, a voltage threshold is set for safe switching of the polarity. This voltage corresponds to a safe temperature differential across the thermoelectric module.

At block 604, polarity switching is initiated based on, for example, a user input.

At block 606, the thermoelectric module is turned off.

At block 608, an adaptive delay is initiated by sampling terminal voltages at the thermoelectric module. These voltages are determined by the temperature difference across the module. Once the magnitude of the voltage is below the threshold established at block 602, the method proceeds to block 610.

At block 610, the applied polarity to the thermoelectric module is reversed, and at block 612, the voltage is applied to the thermoelectric module.

Controlling temperature of a fluid is one useful application for thermoelectric modules. In such an application, the fluid to be temperature-controlled, passes through fluid conduits of a conductive solid body mounted on one plate of the thermoelectric module. Exemplary methods improve the thermal conductance between the thermoelectric modules and the liquid flowing in the fluid conduit.

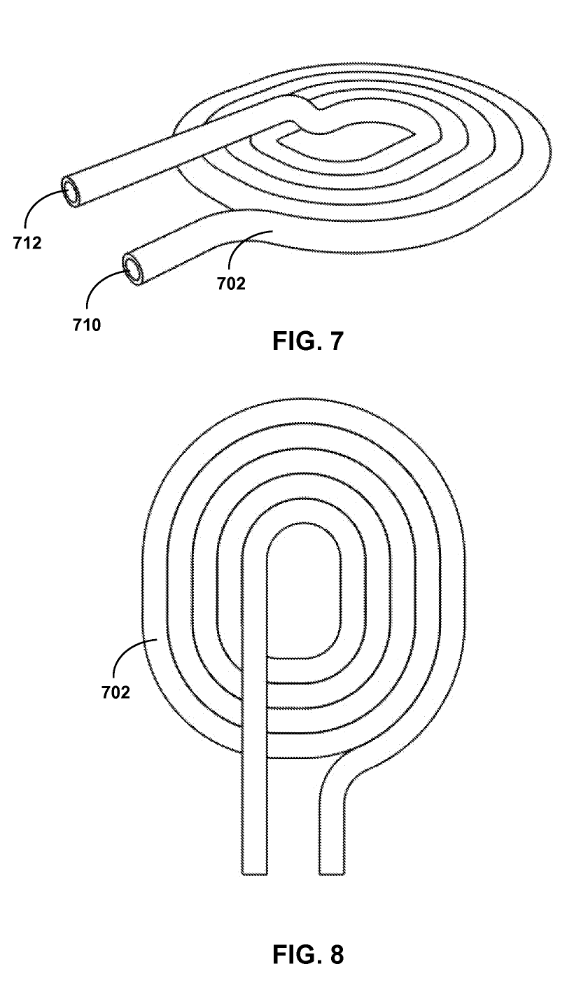

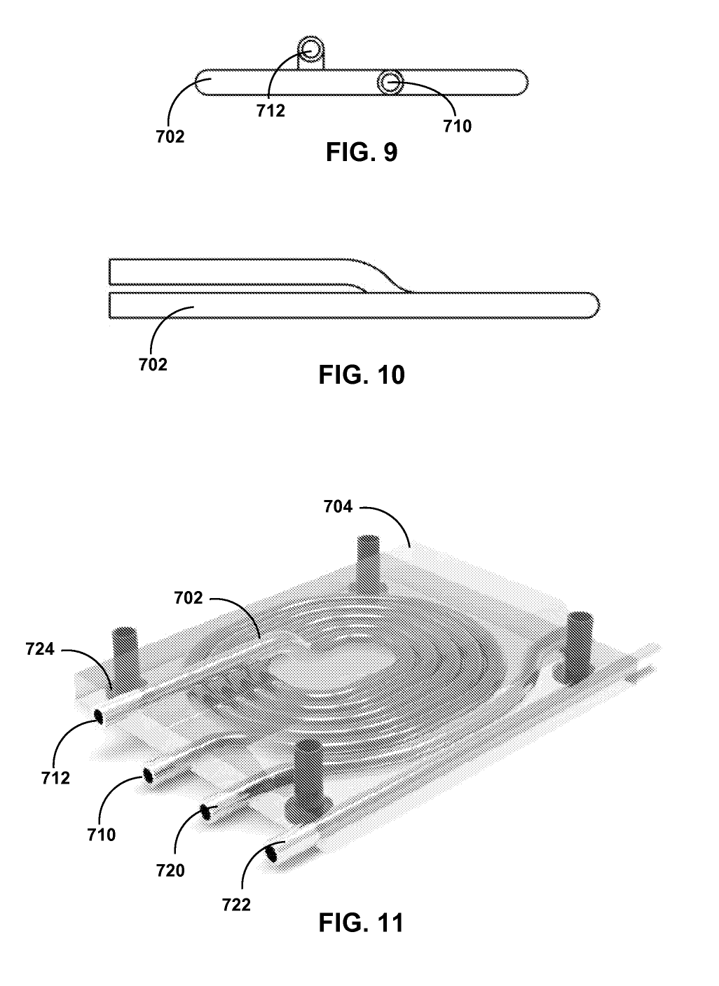

Referring now to FIGS. 7-11, (preferably copper) tubing 702 may be embedded in a (preferably aluminum) block 704 to form an improved pass-through device 700 that improves the temperature conductance from thermoelectric modules to the fluid. In conventional devices, due to minimum bending radius for copper tubing, a great mass of the aluminum casting remained unused. The present disclosure proposes a scrolled pattern of copper embedded in the aluminum casting for the maximum thermal conductance between thermoelectric modules and the fluid. The minimum bending radius, therefore, may only exist at the inner loop, thus maximizing the amount of time the fluid flows through the pass-through device.

FIGS. 7-10 show the copper tubing 702 in perspective, top, front, and side views, respectively. The tubing 702 is wrapped around a central axis and is shown wrapped in an ovular or elongated wrap pattern although a circular pattern is also possible. The fluid openings 710 and 712 are axially offset from one another. The first opening 710 opens along the plane perpendicular to the central axis and that is defined by the wrapped tubing while the second opening 712 opens along a parallel but axially offset plane.

FIG. 11 shows the copper tubing situated inside the block 704. The block may be shaped so as to embed the entirety of the tubing except for the ends of the tube immediately adjacent the openings 710 and 712. In addition to the scroll tubing, two tubing pieces 720, 722 are also embedded in the block 704 and connect the reservoir to the pump and pad. Embedding the two tubing pieces 720, 722 adds additional thermal conductance from the heat exchanger to the fluid. The four shoulder washers 724 may be used to reduce the thermal losses by the clamping screws, which hold the heat-exchanger and heat sink assembly.

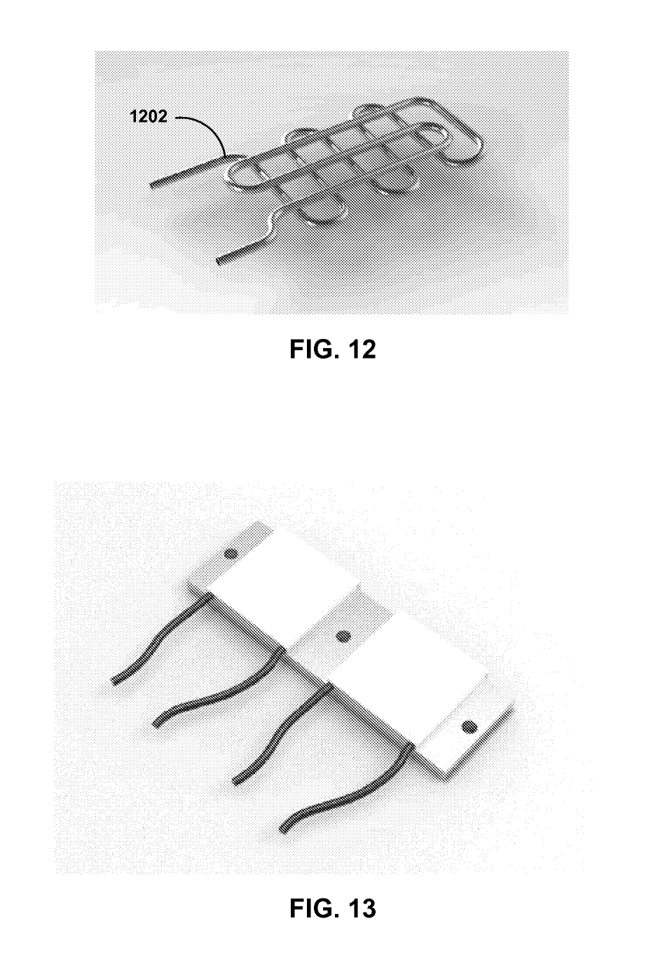

To enhance the thermal conductance between the thermoelectric module and the heat exchanger two or more layers of tubing 1202 patterns embedded in the block may be used, as depicted in FIG. 12. Optionally, the layers may run in transverse directions in relation to each other.

Another exemplary embodiment achieves a high thermal conductance between the thermoelectric modules and the fluid. This method addresses the requirements that are necessary to achieve high thermal transfer from the thermoelectric modules to the fluid, which includes:

a) reduced cumulative mass,

b) reduced height and overall length,

c) extended conduit for fluid flow, and

d) use of thermal compounds with highest thermal conductivity.

The cumulative mass is reduced to lower the heat capacity of the exchanger. The heat capacity is the inertia on the cold plate(s) of the thermoelectric module: the smaller the heat capacity, the lower the inertia, and the higher the ΔT between the cool plate and the hot plate. In practice, the highest ΔT for a thermoelectric module is achieved when there is no heat exchanger mounted on the cold plate (i.e. zero heat capacity).

Reducing the heat capacity reduces the thermal energy necessary to lower the temperature of the cold plate. The thermal energy (Q) necessary to reduce the temperature of a mass by ΔT and thermal capacity (C) for a mass of (m) are linearly related:

Q=C*ΔT (Equation 1)

In the above equation, if the thermal capacity (C) is reduced, the thermal energy (Q) necessary to reduce the temperature by ΔT is also reduced.

The above requirements are addressed in exemplary methods as follows:

1) To reduce the mass that separates the thermoelectric modules and the fluid, the fluid conduits are mechanically machined inside a solid, one piece heat exchanger. For example, the conduits can be milled or drilled in the thickness of the heat exchanger (example details provided below). Mechanical drilling or milling provides the freedom to create any shape or size fluid channel.

2) It is preferred that the fluid conduits are mechanically created in a solid piece of copper, because silver is the only element that has higher thermal conductivity than copper. It is understood that silver is a precious metal and therefore it is cost prohibitive for most practical purposes. It is important to note that other metals such as aluminum can be employed; however, use of copper is preferred as the thermal conductivity of copper is almost twice the thermal conductivity of aluminum.

3) It is preferred to apply a very thin layer of thermal compound between the thermoelectric plates and the heat exchanger. To maximize the thermal transfer, this method uses thermal compound.

4) The heat exchanger preferably has a cross section area equal to the surface area of the thermoelectric modules. Requirements for clamping holes may suggest a heat exchanger having a cross section slightly larger than area of the plates of thermoelectric modules.

The following procedure details how a high efficiency heat exchanger may be built according to this method that also conforms to the general equation for heat transfer by conduction:

Q=k*A*ΔT*t/d (Equation 2)

Where Q is heat transferred by conduction, K is the thermal conductivity of the material, A is the cross sectional area through which heat is transferred, t is the time it takes for the heat transfer, and d is thickness of the material.

It can be seen in (Equation 2) that heat transfer is directly proportional to the cross sectional area A, while the heat capacity in (Equation 1) requires reduced cumulative mass to reduce the necessary thermal energy necessary to generate a ΔT. Therefore, the solid copper is preferably chosen to have the cross section (i.e. top surface) area equal to the total area of the ceramic plates of the TECs or slightly larger to accommodate for mechanical clamping holes as shown in FIG. 13. It can be seen that the width of the copper plate is the same as the width of the thermoelectric modules while there is sufficient space intended for three mechanical holes and shoulder washers that isolate the clamping screws from the copper plate.

The copper plate shown in FIG. 13 is intended for two thermoelectric modules. It is understood that a heat exchanger according to exemplary methods can accommodate one or more thermoelectric modules or can employ any number of mechanical clamping holes.

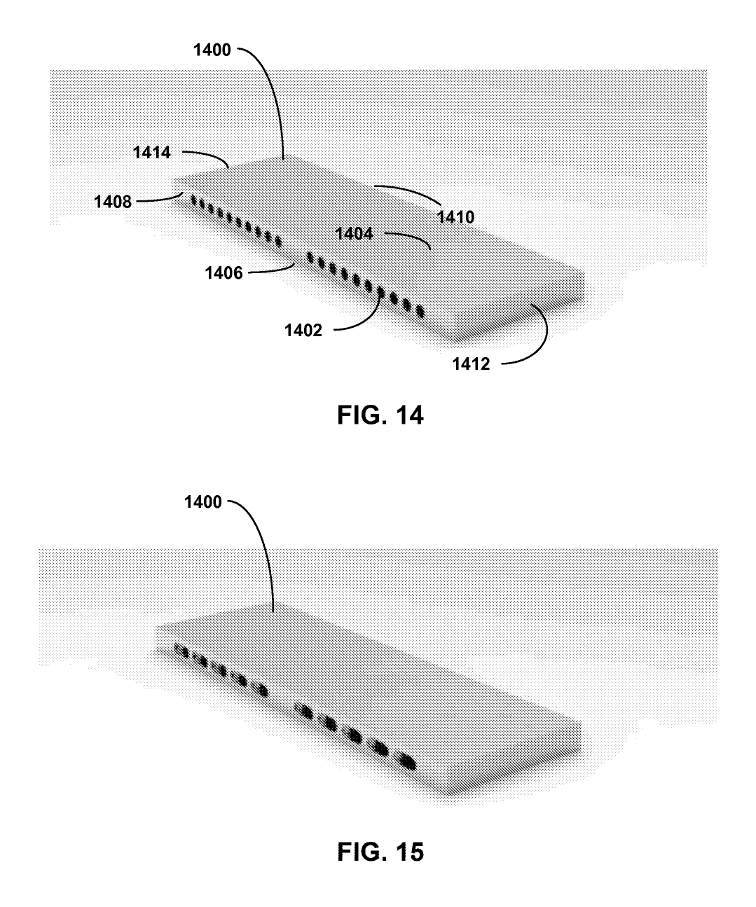

The thickness of the copper plate is chosen according to the diameter or the height (for rectangular channels) of the fluid channels that will be mechanically created in the copper plate. The diameter of the fluid channel is designed according to the flow/pressure requirements for the fluid and the pump specifications. It can be seen in (Equation 2) that, to achieve the maximum possible thermal conduction and to eliminate seams, the fluid channels are mechanically machined in the thickness of copper as shown in FIG. 14. For example, twenty through-holes 1402 made in the thickness of copper plate 1400 will be used to form channels for fluid flow. The plate 1400 has top and bottom surfaces 1404 and 1406 machined flat for interfacing with adjacent components. The holes 1402 are drilled into long sides 1408, 1410 of the plate and extend through the width of the plate parallel to the short sides 1412, 1414 of the plate.

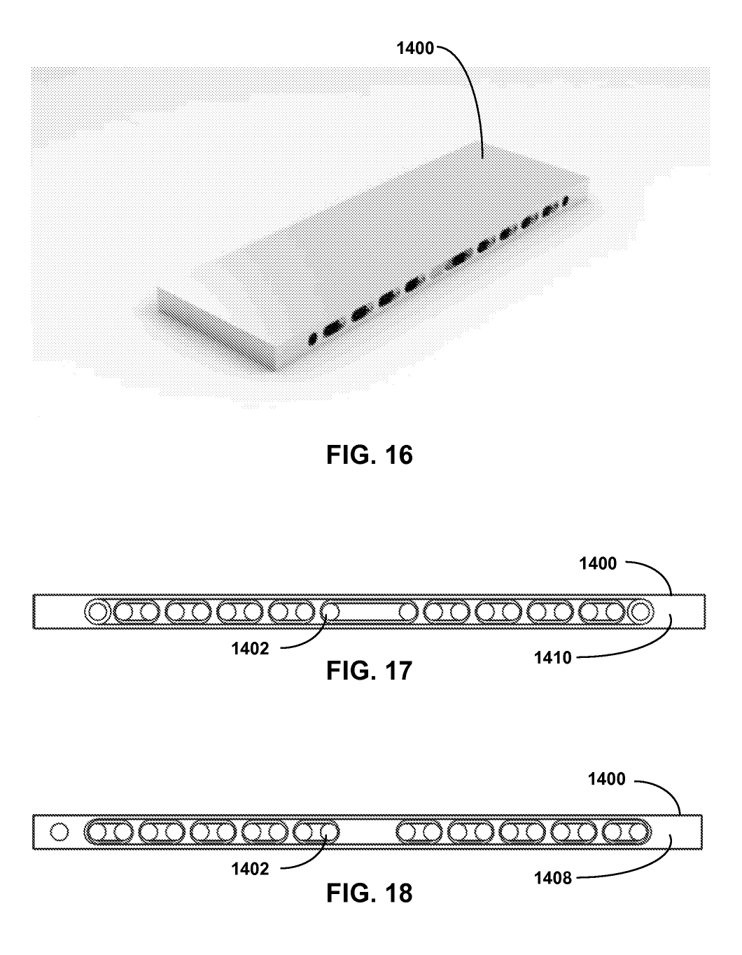

Adjacent holes are connected by side-milling as shown in FIG. 15. The milling pattern is slightly different on the opposite side of the drilled copper plate as shown in FIG. 16.

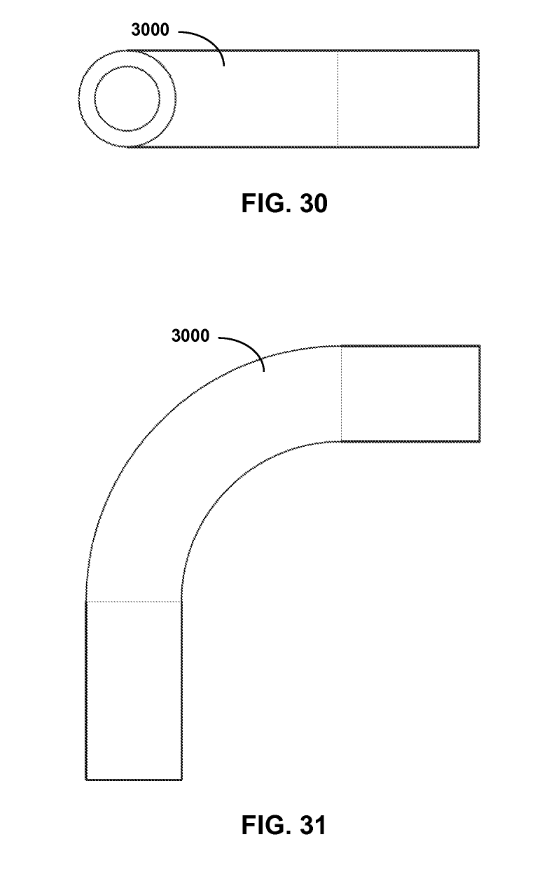

As can be seen in FIG. 16, the leftmost and rightmost holes are not machined. These openings are the inlet and outlet of the plate and may be fitted with tubing, for example the copper tubing 3000 shown in FIGS. 30 and 31. These copper tubes may then be used as nubs to attach flexible tubing to. These copper nubs are pre-bent and to provide rigidity to the flexible tubing that prevents kinking at the bend of the tubing.

The finished machined block can be seen clearly in several views in FIGS. 17-23.

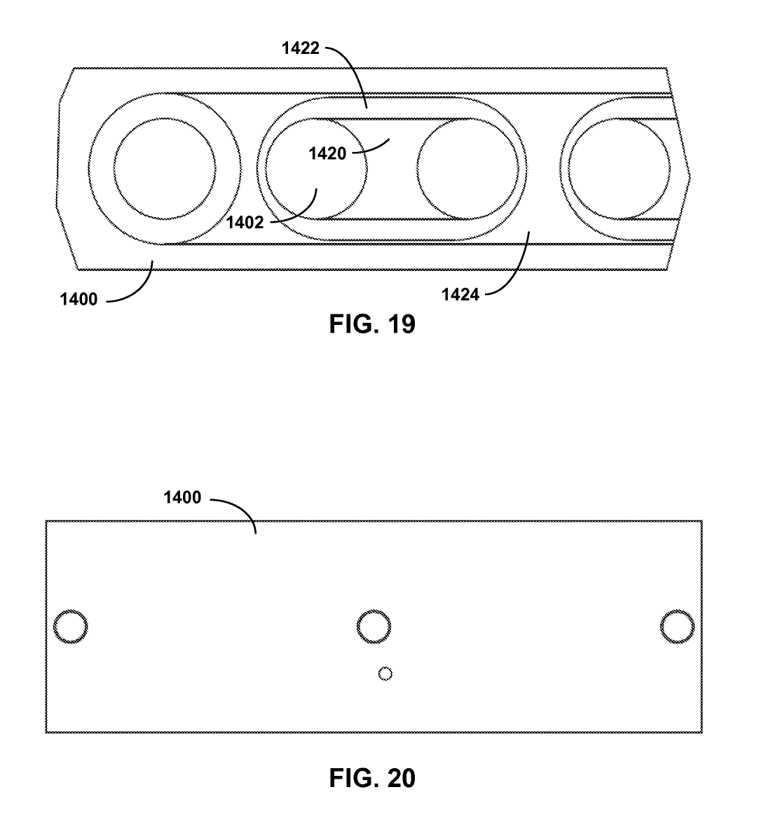

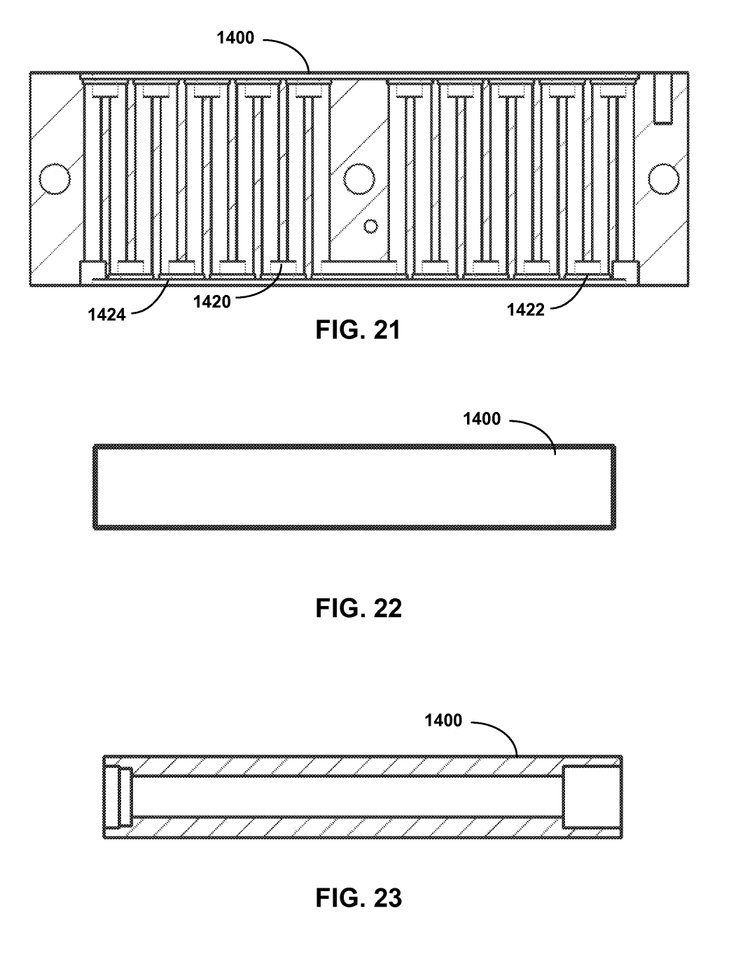

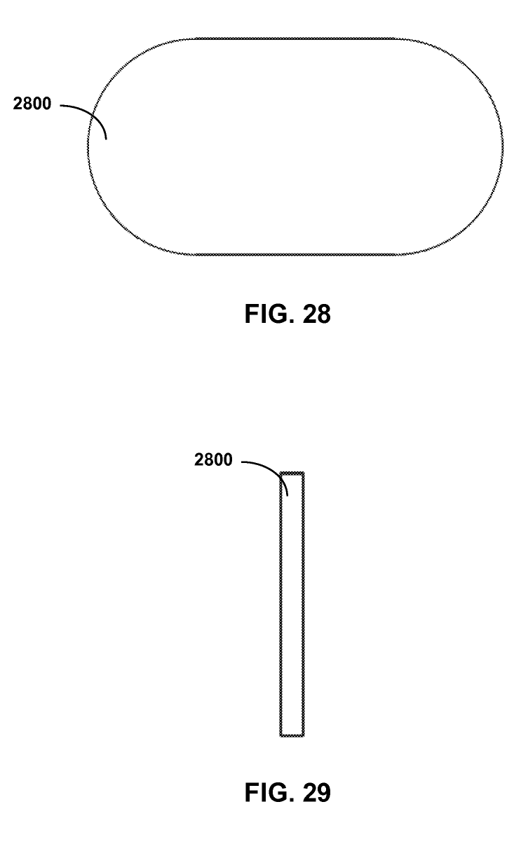

FIGS. 17 and 18 show end views of the plate 1400 with the holes 1402 drilled into the long edges of the plate. Adjacent holes 1402 are fluidly connected with each other by side milling as described above and a detailed view of connected holes is shown in FIG. 19. As shown, a land 1420 is created between adjacent holes 1402 that defines the inside bend of the fluid passage. This land may also be seen in FIG. 21. Another side milling operation, shallower than the other, produces a land 1422 around the adjacent holes. This land provides a location for a plug 2800 to be inserted to define the outside bend of the passageway through the plate. An example plug is shown in FIGS. 28 and 29, FIG. 28 showing the plug 2800 in front view and FIG. 29 in side view. This plug may be shaped to fit the land 1422 and is preferably an ovular shape. The plug may be made from any appropriate material, but is preferably fiberglass. Fiberglass may be epoxied to the plate 1400 and may provide insulation and increase efficiency of the plate by reducing heat transfer through the side of the plate. The plugs may be held in by epoxy that is spread along the land 1424 created in a third side milling operation that runs along the length of the long sides from the first to the last hole.

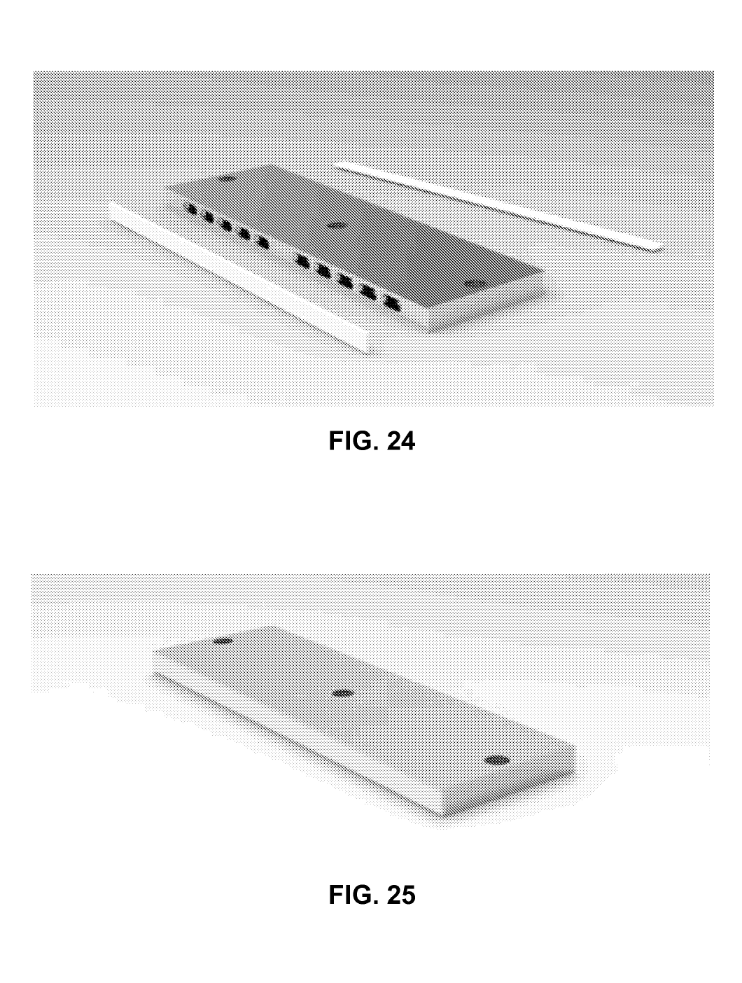

Alternatively, as shown in FIGS. 24-27, the two sides of the copper plate may be covered, for example, by two thin flat strips of copper (or other material), to form a continuous fluid channel therebetween. Although of easier construction, this method may have more issues creating a tight seal between each of the adjacent fluid passages.

FIG. 24 shows the machined copper plate and two thin strips of fiberglass for covering the side channels. It is preferred that non-conductive side strips are epoxied to the copper plate, as shown in FIG. 25. It is possible to solder thin strips of copper to the sides of the copper plate, however, high temperature of soldering can cause deep oxidization on the surface of conduits which reduces the thermal conductance. Using fiber glass material will cause no oxidization of copper plate.

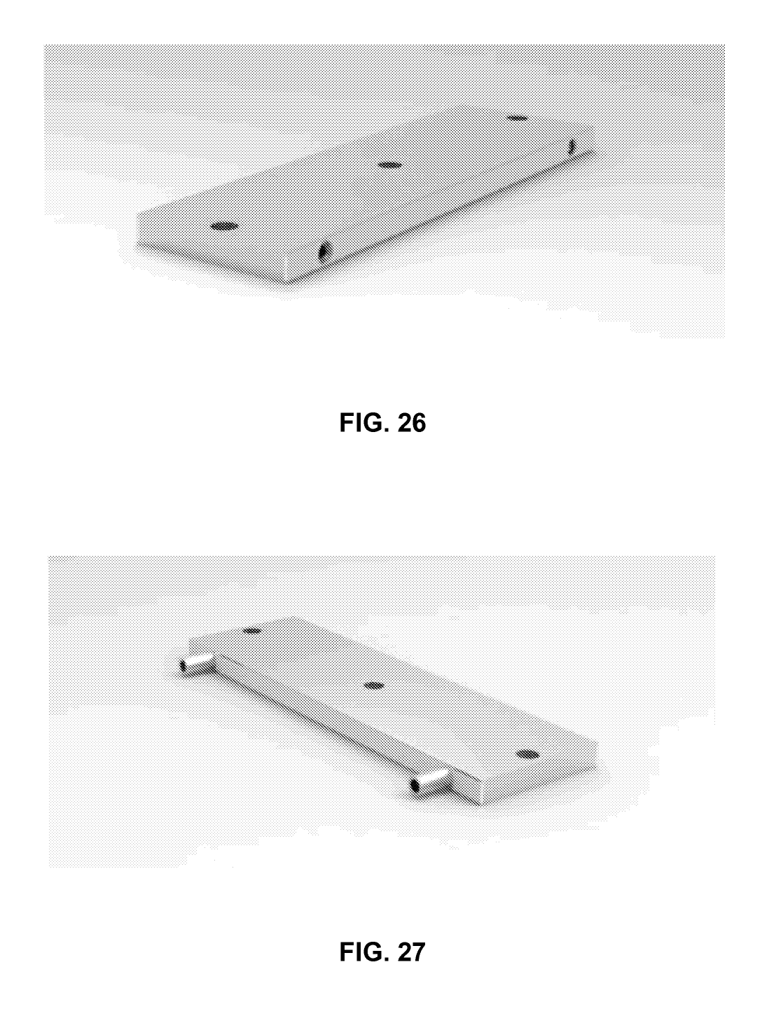

In order to enable the fluid flow into and out of the copper exchanger, two short pieces of tubing are attached to the inlet and outlet holes as shown in FIGS. 26 and 27. These tubes may be straight as shown, or curved as depicted in FIGS. 30 and 31.

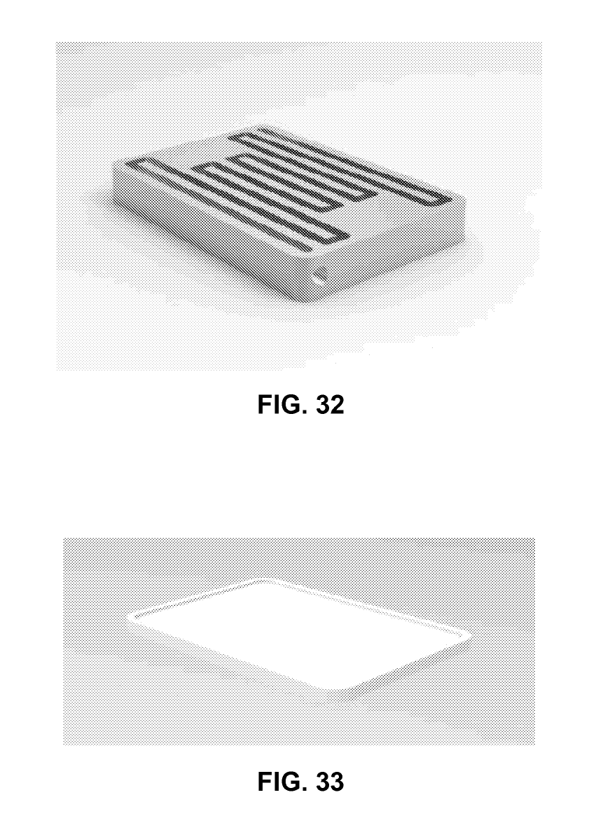

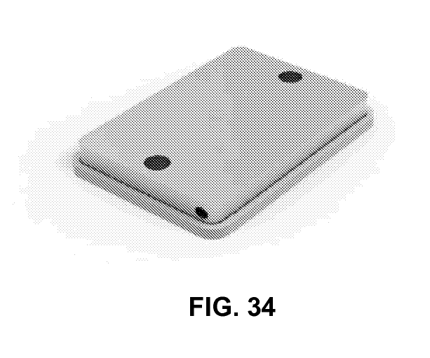

As shown in FIG. 32, it is also possible to mill the fluid conduit in the copper by surface milling of the copper plate. The plate may be covered on the milled surface by a (preferably non-conductive) plug such as fiber glass as shown in FIGS. 33 and 34.

Although surface milling is less complicated and less expensive than drilling in the thickness of copper, it has lower performance due to the relatively large seam between the plate that covers the milled copper plate. Again, soldering or brazing a copper plate is possible although it reduces the thermal performance due to oxidization.

The high efficiency thermal exchanger may be used in exemplary thermal therapy devices as discussed above, but it may also be used in a number of other applications such as:
Power transistor, semiconductor, and CPU cooling,
Cooling of laser diodes and related circuits, and
Cell culture and microscope stages that require small, efficient thermal exchangers.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A thermal therapy system comprising:
a tissue-interacting device to provide thermal therapy to a structure of a therapy-receiving person's body;
a fluid-manipulating device which heats/cools the therapy-providing fluid including a pump for motivating the circulation of the therapy-providing fluid through the system and a heat exchanger for heating/cooling the therapy-providing fluid;
tubing, and associated fittings, between the tissue-interacting device and the fluid-manipulating device; and
an electrical control for controlling the temperature of the therapy-providing fluid,
wherein the heat exchanger comprises a heat sink, a block through which the therapy-providing fluid flows, and one or more thermoelectric devices placed in thermal contact with the sink and the block,
wherein the block includes a monolithic plate having a fluid passage defined therein by machined surfaces therein and by one or more plugs.

2. The thermal therapy system of claim 1, wherein the one or more plugs are fiberglass.

3. The thermal therapy system of claim 1, wherein the one or more plugs are epoxied to the plate.

4. The thermal therapy system of claim 1, wherein the fluid passage is serpentine and the one or more plugs fit into respective lands machined into side surfaces of the plate and define outer bends of the serpentine passage.

5. The thermal therapy system of claim 1, wherein the monolithic plate is copper.

6. The thermal therapy system of claim 1, wherein the serpentine fluid passage has a circular cross-section.

7. A block for a heat exchanger comprising:
a monolithic plate having a serpentine fluid passage contained therein, the fluid passage having a straight portion with sidewalls defined by machined inner surfaces of the plate and a bend portion with sidewalls defined by machined inner surfaces of the plate and a sidewall defined by a plug.

8. The block of claim 7, wherein the plug is fiberglass.

9. The block of claim 7, wherein the plug is epoxied to the plate in a machined recess of a long edge of the plate.

10. The block of claim 7, wherein the monolithic plate is copper.

11. The block of claim 7, wherein the straight portion of the serpentine fluid passage has a circular cross-section.

12. The block of claim 7, wherein the sidewall defined by the plug is on an exterior bend of the bend portion.

13. A method of making a block for a heat exchanger, the method comprising:
drilling a series of through-holes into monolithic plate, the through-holes extending from a first edge of the plate to an opposite second edge of the plate;
side milling a first recess in one of the edges between two adjacent through-holes, the recess having a first depth;
side milling a second recess in the same one of the edges between and around the two adjacent through-holes, the recess having a second depth and the second depth being shallower than the first depth; and
fitting a plug into the second recess and adhering the plug to the plate.

14. The method of claim 13, further comprising:
side milling a third recess in the same one of the edges around the second recess, the third recess having a third depth, the third depth being shallower than the second depth.

15. The method of claim 13, wherein the edges are long edges of the plate, and wherein the through-holes extend parallel to short edges of the plate.

16. The method of claim 13 further comprising:
fitting rigid tubing nubs to inlet and outlet openings of the plate.

17. The method of claim 13, wherein the plug is fiberglass.

18. The method of claim 13, wherein the monolithic plate is copper.

19-26. (canceled)
